(19) United States
(12) Patent Application Publication
(10) Pub. No.: US 2026/0114889 A1
(43) Pub. Date: Apr. 30, 2026

(54) INTEGRATED MECHANICAL THROMBECTOMY AND ASPIRATION DEVICE PRODUCED FROM A SINGLE ELEMENT

(71) Applicant: Covidien LP, Mansfield, MA (US)

(72) Inventors: Ravikant Sadasivuni, Aliso Viejo, CA (US); Danyong Zeng, Aliso Viejo, CA (US); Tejashri Kumar, Fountain Valley, CA (US)

(21) Appl. No.: 19/366,847

(22) Filed: Oct. 23, 2025

Related U.S. Application Data
(60) Provisional application No. 63/711,607, filed on Oct. 24, 2024.

(51) Int. Cl. A61B 17/221; A61B 17/22

(52) U.S. Cl. CPC A61B 17/221; A61B 2017/22041; A61B 2017/22079; A61B 2017/2215

(57) ABSTRACT

Systems and methods for removing obstructions from bodily lumens are disclosed herein. A device for removing an obstruction may comprise a tubular body defining a lumen fluidically coupled to a suction source. The device may further comprise an aspiration region comprising a plurality of sidewall openings in fluid communication with the lumen and a mechanical engagement region disposed distal to the aspiration region. The mechanical engagement region may comprise an expandable member configured to expand radially outward. In some aspects, the expandable member is integrally formed with the tubular body.

### CROSS-REFERENCE TO RELATED APPLICATIONS

This application claims the benefit of and priority to U.S. Provisional Patent Application Ser. No. 63/711,607 filed Oct. 24, 2024, the entire disclosure of which is incorporated by reference herein.

### TECHNICAL FIELD

The present technology relates to tubular devices having longitudinally arranged and fluidically connected openings at a distal end region. The present technology also relates to tubular devices having expandable structures for improved clot retrieval.

### BACKGROUND

Ischemic strokes are caused by the interruption of blood supply to the brain. For example, the blood supply may be interrupted by a thrombus (e.g., a blood clot) lodged in an artery responsible for feeding oxygenated blood to the brain. If the disruption in blood supply occurs for a sufficient amount of time, the continued lack of nutrients and oxygen causes irreversible cell death, potentially leading to permanent neurological deficit or death. Therefore, immediate restoration of blood flow is critical. One method of restoring blood supply to the brain involves removing the thrombus via mechanical thrombectomy, including stent-retriever thrombectomy and direct aspiration applied to the proximal end of the thrombus by the distal end of an aspiration catheter.

In order to restore blood supply in a timely manner, it has been found highly beneficial to fully remove the clot in an initial attempt, also referred to herein as a first pass. A first pass removal of a clot has been correlated to better clinical outcomes, and is referred to herein as the “first pass effect.” However, current methods of mechanical thrombectomy and direct aspiration do not provide means for adequately capturing clot material that may become fragmented or disengaged from the retrieval device during the initial attempt, thereby requiring additional passes. Accordingly, there is a need for systems, devices, and methods for addressing the problems noted above in order to increase the likelihood of achieving the first pass effect and avoiding the escape of clot material into distal vasculature.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1 shows a perspective view of a thrombectomy system in accordance with some embodiments of the present technology.

FIG. 2 is a schematic perspective view of a distal region of a thrombectomy device in accordance with some embodiments of the present technology.

FIGS. 3A and 3B are side schematic cross-sectional views showing a one-way valve before and after passage of a guidewire in accordance with some embodiments of the present technology.

FIG. 4 is a schematic side view of a distal region of a thrombectomy device comprising two tubular bodies in accordance with some embodiments of the present technology.

FIG. 5A is a schematic side view of the distal region of FIG. 4 with the expandable member in a collapsed state.

FIG. 5B is a schematic side view of the distal region of FIG. 4 with the expandable member in an expanded state in which openings in the inner and outer tubular bodies are substantially aligned.

FIGS. 6-8 are schematic views of various embodiments of distal regions of thrombectomy devices in accordance with the present technology.

FIGS. 9A-9D illustrate exemplary methods of removing clot material from a blood vessel lumen using a thrombectomy system in accordance with the present technology.

### DETAILED DESCRIPTION

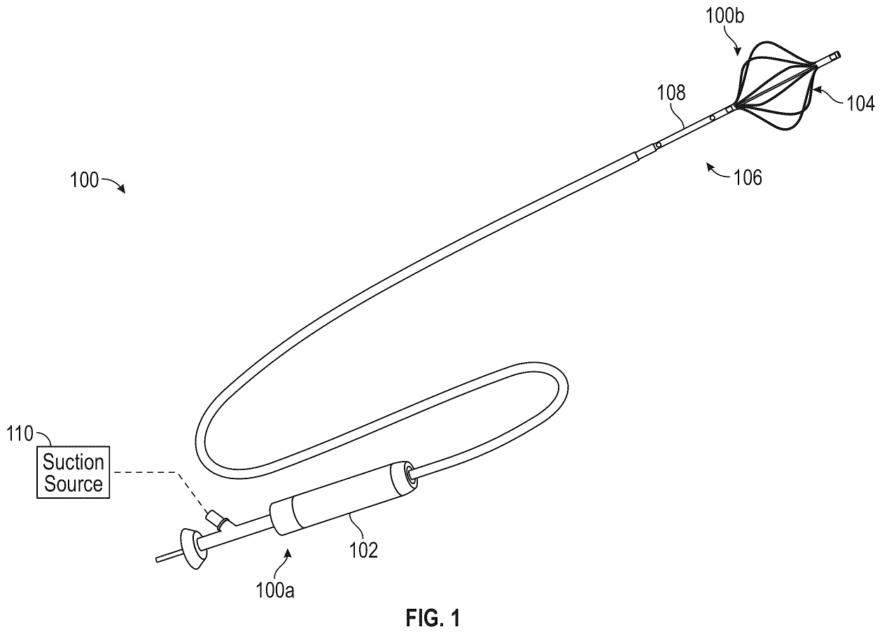

The present technology relates to systems, devices, and methods for treating vascular obstructions, such as vessel occlusions. In some embodiments, a device includes a tubular structure having distally arranged sidewall openings. In some embodiments, the device includes one or more expandable structures to, for example, prevent leakage of clot material to distal vasculature, referred to elsewhere herein as “distal protection.” Specific details of several embodiments of the technology are described below with reference to FIGS. 1-9D.

The detailed description set forth below is intended to describe various configurations of the subject technology and is not intended to represent the only configurations in which the subject technology may be practiced. The detailed description includes specific details for the purpose of providing a thorough understanding of the subject technology. However, it will be clear and apparent to those skilled in the art that the subject technology is not limited to the specific details set forth herein and may be practiced using one or more implementations.

As noted previously, when attempting to remove a blood clot to restore blood supply, it is generally advantageous to fully remove the clot in the first pass. Partial or complete loss of the clot material during retraction of the retrieval device can necessitate further access to the treatment site, creating significant delays in the procedure as the treatment system must be renavigated to the treatment site(s) to make a subsequent pass and additionally creates added trauma to the arteries through which the retrieval device is again or newly passed. However, first-pass success is difficult to achieve using current methods, especially in particular applications. In some instances, a clot may break into small fragments during a thrombectomy procedure, which may not all be adequately captured by a stent retriever. In other instances, a clot may be too large or firmly lodged within a blood vessel lumen to be retrieved by aspiration alone. Accordingly, it is beneficial to combine these methods to achieve more efficient and complete clot removal.

However, particularly in the tortuous anatomy of the cranial vasculature, it can be cumbersome to effectively navigate multiple devices, for example, a stent retriever and an aspiration catheter, to a treatment site. Providing a mechanical engagement element and a means for aspiration in a single device facilitates access to clot material located in such distal vasculature. Additionally, this configuration allows for smoother passage through the vasculature, reducing the risk of captured clot material becoming detached from the device during removal.

Embodiments of the present technology relate to systems for removing clot material from bodily lumens comprising a mechanical engagement region and an aspiration region integrated within a single device. In some embodiments, the mechanical engagement region comprises an expandable member integrally formed with a tubular body. The tubular body may have an outer diameter that is substantially constant at least along the mechanical engagement region, the aspiration region, and/or an entire distal region when the expandable member is in a collapsed configuration. In some embodiments, the expandable member acts as a distal protection element to prevent migration of clot material away from the device. In some embodiments, the device comprises a distal plug such that aspiration is applied through sidewall openings in the tubular body and not through the distal end of the device.

FIG. 1 illustrates an integrated mechanical and aspiration thrombectomy system in accordance with some embodiments of the present technology. The thrombectomy system can include a treatment device 100 having a proximal portion 100a configured to be coupled to at least one extracorporeal element and a distal portion 100b configured to be intravascularly positioned within a blood vessel lumen at a treatment site. In some embodiments, the blood vessel is an intracranial vessel. The treatment site may be proximate a thrombus, distal to a thrombus, within a thrombus, or any suitable location. The device 100 includes a handle 102 at the proximal portion 100a, a mechanical engagement region 104 and/or an aspiration region 106 at the distal portion 100b, and at least one tubular body 108 extending therebetween. In some embodiments, the mechanical engagement region 104 is disposed distally of the aspiration region 106 along the tubular body 108. In some embodiments, the mechanical engagement region 104 is disposed proximally of the aspiration region 106. In some embodiments, the mechanical engagement region 104 partially or entirely overlaps with the aspiration region 106.

In some embodiments, the at least one extracorporeal element includes a suction source 110 coupled to the proximal portion 100a of the device 100 in fluid communication with the tubular body 108. In embodiments comprising multiple tubular bodies 108, the suction source 110 may be in fluid communication with one or more of the multiple tubular bodies 108. The suction source 110 may be configured to supply negative pressure to the treatment site through the aspiration region 106, thereby engaging clot material. While the embodiment illustrated by FIG. 1 shows a suction source 110 coupled to the device 100, it should be appreciated that other elements (e.g. a fluid source configured to deliver a therapeutic agent, an electrical element configured to supply a medically useful current, etc.) may additionally or alternatively be coupled to the device 100.

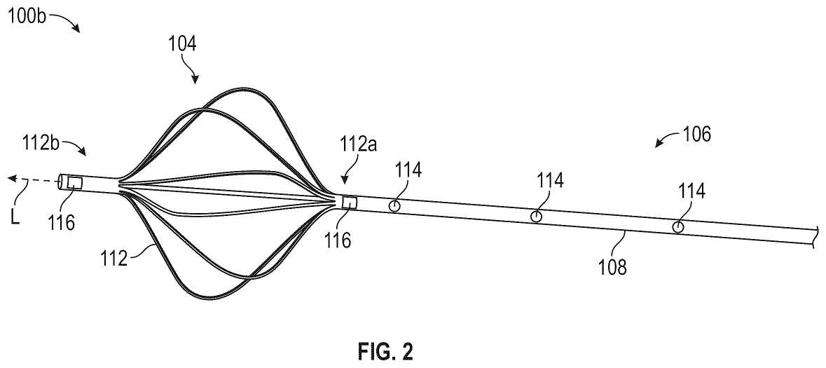

FIG. 2 shows a view of the distal portion 100b of device 100 according to some embodiments of the present technology. In the embodiment of FIG. 2, the mechanical engagement region 104 is positioned distally of aspiration region 106 along the tubular body 108; however, it should be appreciated that other configurations are possible.

The mechanical engagement region 104 may comprise an expandable member 112 configured to expand into engagement with clot material at a treatment site. The expandable member 112 is shown in an expanded configuration in FIG. 2; however, the expandable member 112 may also comprise a low-profile, collapsed configuration to, for example, facilitate navigation of the device 100 through the vasculature. In some embodiments, the expandable member 112 self-expands from the collapsed configuration to the expanded configuration when in position at a treatment site. The expandable member 112 may be made of a shape memory material, and may be heat-set to a desired shape (e.g., a radially expanded configuration). While transitioning from the collapsed to the expanded configuration, the expandable member 112 may foreshorten such that a relative distance between a first end 112a and a second end 112b is reduced along a longitudinal axis L of the tubular body 108.

In some embodiments, the mechanical engagement region 104 is integrally formed with the tubular body 108 such that the tubular body is 108 is unencumbered by any welds, collars, joints, or other coupling means that would be necessary to attach a separate mechanical engagement element to the device 100. Accordingly, the distal portion 100b is capable of maintaining a substantially constant outer diameter along its length when the expandable member 112 is in the collapsed configuration. This provides the device 100 with greater flexibility in navigating tortuous anatomy, as well as greater efficiency during manufacturing. The expandable member 112 may comprise a plurality of filaments separated by a plurality of slits cut into the sidewall of the tubular body 108, providing a unitary structure. In some embodiments, the unitary structure is achieved by laser cutting the device from a single element (e.g., a single tubular member such as a hypotube can have slits cut into its sidewalls to define the mechanical engagement region 104). When the expandable member 112 transitions to the expanded state, the plurality of filaments may flex away from one another. While this and other features are discussed regarding device 100, it should be appreciated that this feature may be applied to any of the embodiments discussed herein.

The aspiration region 106 may comprise one or more sidewall openings 114 configured to supply negative pressure to the treatment site. The sidewall openings 114 may be spaced apart from one another along a longitudinal axis L of the tubular body 108 and/or radially spaced about an outer surface of the tubular body 108. While the sidewall openings 114 are shown substantially in radial alignment in FIG. 2, other configurations are possible. In some embodiments, the sidewall openings 114 are radially displaced about the tubular body 108 at an angle; for example, an angle of 45, 90, 135, or 180 degrees. The sidewall openings 114 illustrated in FIG. 2 are substantially circular, but in some embodiments, may be shaped as ovals, ellipses, slits, or any suitable geometry. In some embodiments, the sidewall openings 114 are formed with geometries configured to assist with clot material engagement (e.g. star shapes, slit shapes, ridge shapes, etc.). Additionally, the sidewall openings 114 may be sized between 5% and 100% of the outer diameter (OD) of the tubular body 108. In some embodiments, the sidewall openings 114 may each have an opening size no more than about 90%, 80%, 70%, 60%, 50%, 40%, 30%, 20%, or 10% of the OD of the tubular body 108. The sidewall openings 114 may be of substantially the same size, or they may comprise different sizes. For example, in some embodiments, the sizes of the sidewall openings 114 decrease in a proximal to distal direction along the length dimension of the device. This may advantageously provide optimal suction force and minimize escape of clot material into the vasculature.

In some embodiments, the tubular body 108 comprises an OD from about 0.010 in to 0.050 in. In some embodiments, the tubular body 108 comprises an OD from 0.017 in to 0.045 in. The tubular body may comprise any suitable OD, for example, less than about 0.010 in, about 0.010 in, about 0.020 in, about 0.030 in, about 0.040 in, about 0.050 in, or greater than about 0.050 in.

At least a portion of the device 100 may be formed from a metallic material. In some embodiments, the metallic material comprises a shape memory alloy, Nitinol, and/or stainless steel. For example, at least one of the tubular body 108, the mechanical engagement region 104, the aspiration region 106, and/or the expandable member 112 may be formed from the metallic material. In some embodiments, at least a portion of the device 100 comprises a polymer coating, for example, a Pebax coating. At least one of the tubular body 108, the mechanical engagement region 104, the aspiration region 106, and/or the expandable member 112 may comprise the polymer coating. In some embodiments, the polymer coating comprises a hydrophilic coating. In some embodiments, the polymer coating is configured to ease navigation of the device 100 through a patient's vasculature and/or to enhance biocompatibility at the treatment site.

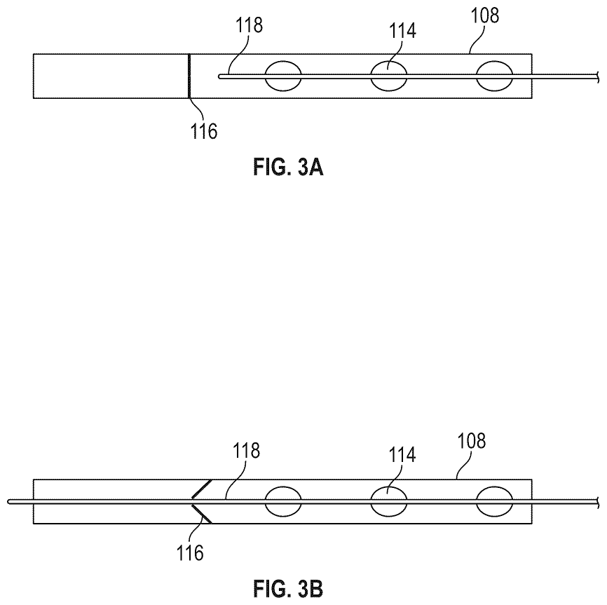

In certain embodiments such as the embodiment of FIG. 2, the distal portion 100b may include at least one distal plug 116 configured to prevent aspiration through the distal end of device 100. Additionally or alternatively, the tip of distal portion 100b may be shaped with outer and inner diameters sufficiently small such that the device may pass over a guidewire 118 while minimizing the escape of clot material. As shown in further detail in FIGS. 3A and 3B, the distal plug 116 may comprise a one-way valve designed to permit the tubular body 108 to be slidably advanced over a guidewire 118, aiding navigation during placement of the device 100.

FIG. 3A shows the guidewire 118 prior to advancement through the one-way valve. In this configuration, the flow of fluid is inhibited through the one-way valve such that no aspiration is provided through the distal end of device 100. As shown in FIG. 3B, as the guidewire 118 is slidably advanced, the one-way valve is designed to allow passage of the guidewire 118 without any leakage of physiological fluids into the lumen of the tubular body 108. Alternatively, the one-way valve can remain closed in the absence of a guidewire 118 or may open only when a guidewire 118 is introduced. In some embodiments, the device 100 includes a single plug 116 or a plurality of plugs 116. One or more plugs 116 may be positioned distally of the aspiration region 106, proximally of the mechanical engagement region 104, and/or distally of the mechanical engagement region 104. In some embodiments, one or more plugs 116 may be positioned at a distalmost end of the device 100. The distalmost plugs 116 may remain closed under aspiration. In some embodiments, one or more plugs 116 positioned proximally of the distalmost plug 116 may open under aspiration to provide negative pressure at the sidewall openings 114, and in other embodiments, all of the plugs 116 may remain closed. In some embodiments, the one or more plugs 116 are radiopaque plugs.

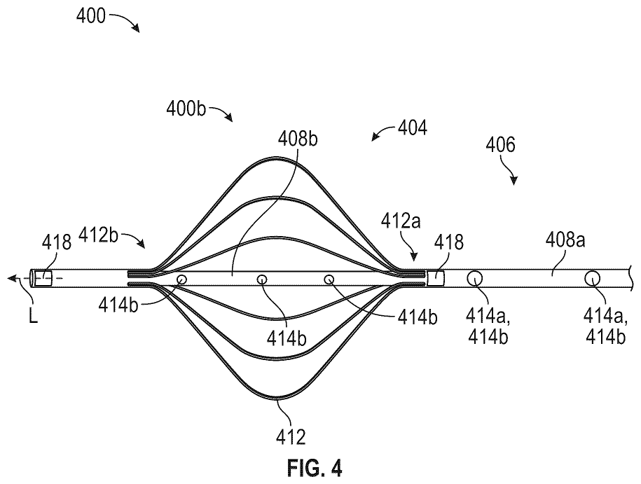

FIG. 4 shows a side view of the distal portion 400b of a device 400 according to some embodiments of the present technology. The embodiment of FIG. 4 is generally similar to that of FIG. 2, differing in that the device 400 comprises an outer tubular body 408a and an inner tubular body 408b slidably disposed therein. In some embodiments, the outer tubular body 408a comprises a first plurality of sidewall openings 414a and the inner tubular body 408b comprises a second plurality of sidewall openings 414b. As will be described in greater detail herein, the first and second plurality of sidewall openings may be substantially aligned when the expandable member 412 is in the expanded configuration at a treatment site. In some embodiments, the aspiration region 406 comprises the first and second plurality of sidewall openings 414a, 414b. The aspiration region 406 may overlap with the mechanical engagement region 404 such that negative pressure is supplied to the treatment site from a location within the expandable member 412 and/or from a location proximate of or distal to the expandable member 412. One or more plugs 418 may be provided such that the inner lumens of outer tubular body 408a and inner tubular body 408b are substantially fluidly sealed from physiological fluids. In such a configuration, aspiration can be provided through at least one of the first and second plurality of sidewall openings 414a, 414b and not through the distal end of the device 400. In some embodiments, aspiration is provided to the treatment site only when the first and second plurality of sidewall openings 414a, 414b are substantially aligned.

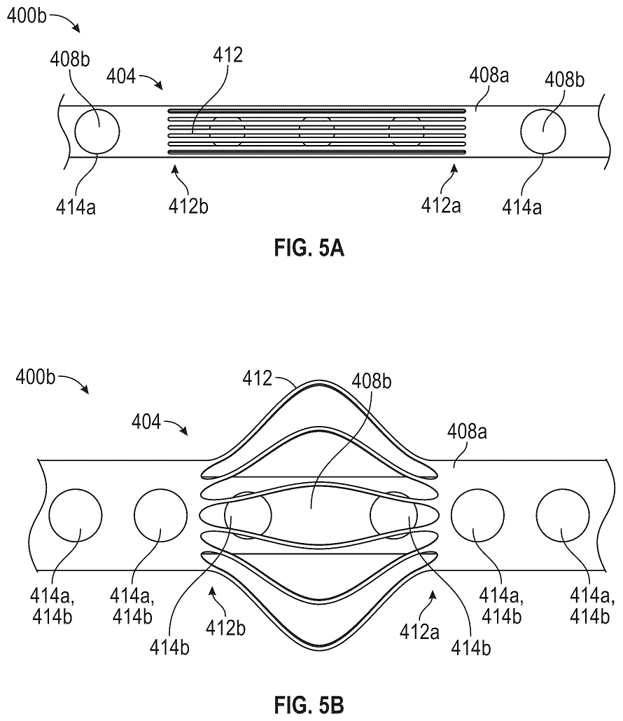

FIGS. 5A and 5B are schematic views of the distal portion 400b of device 400 in the compressed state (FIG. 5A) and the expanded state (FIG. 5B). As can be seen in FIG. 5A, the first plurality of sidewall openings 414a of outer tubular body 408a may be substantially blocked by a sidewall of inner tubular body 408b when the expandable member 412 is in the compressed state. In some embodiments, the expandable member 412 is integrally formed with the outer tubular body 408a and maintains a substantially constant outer diameter along a length of the distal portion 400b in the compressed state. In some embodiments, the expandable member 412 is formed from a plurality of longitudinal slits cut into the sidewall of the outer tubular body 408a.

FIG. 5B shows the mechanical engagement region 404 after the expandable member 412 has been transitioned from the compressed state to the expanded state. In the expanded state, the first plurality of sidewall openings 414a and the second plurality of sidewall openings 414b may be substantially aligned. In some embodiments, the alignment of the first and second plurality of sidewall openings 414a, 414b is permitted by relative axial motion between the outer and inner tubular bodies 408a, 408b. Outer tubular body 408a may be configured to foreshorten when deployed at a treatment site, causing a relative distance between the first end 412a and the second end 412b of the expandable member 412 to decrease. Such foreshortening may cause the expandable member 412 to radially expand, thereby exposing at least some of the second plurality of sidewall openings 414b to physiological fluids at the treatment site. Additionally or alternatively, at least some of the second plurality of sidewall openings 414b may be exposed to the treatment site via alignment with at least some of the first plurality of sidewall openings 414a.

In some embodiments, the first and second plurality of sidewall openings 414a, 414b are aligned such that fluid communication is provided between the treatment site and a lumen of the first tubular body 408a and/or a lumen of the second tubular body 408b. Accordingly, negative pressure from a suction source may be blocked when expandable member 412 is in the compressed state shown in FIG. 5A and supplied to the treatment site when expandable member 412 is in the expanded state shown in FIG. 5B. In some embodiments, at least some of the second plurality of sidewall openings 414b can be only partially exposed to physiological fluids at the treatment site during and/or after transition of the expandable member 412 from the compressed state to the expanded state.

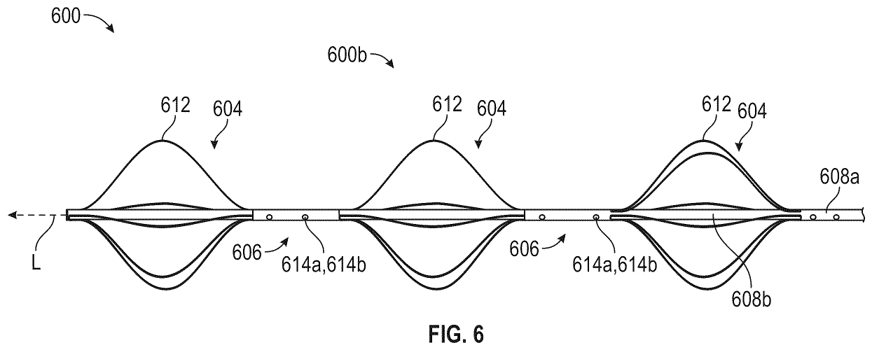

FIG. 6 shows a side view of the distal portion 600b of a device 600 according to some embodiments of the present technology. In such embodiments, the mechanical engagement region 604 may be a plurality of separate mechanical engagement regions 604, each comprising an expandable member 612. In some embodiments, at least one of the expandable members 612 is formed integrally with the device 600 as described previously herein. The distal portion 600b may also comprise a plurality of aspiration regions 606 disposed between, distally, and/or proximally of one or more of the plurality of mechanical engagement regions 604. In some embodiments, the device 600 comprises an outer tubular body 608a and an inner tubular body 608b. Alternatively, the device 600 comprises only an outer tubular body 608a. The outer tubular body 608a and/or inner tubular body 608b may contain a first and second plurality of sidewall openings 614a, 614b which may be configured to substantially align when the expandable members 612 are in the expanded state. In some embodiments, the second plurality of sidewall openings 614b are spaced such that they only overlap with the first plurality of sidewall openings 614a at one or more aspiration regions 606 between the mechanical engagement regions 604. In this configuration, aspiration is not supplied through the expandable members 612; however, other configurations are possible.

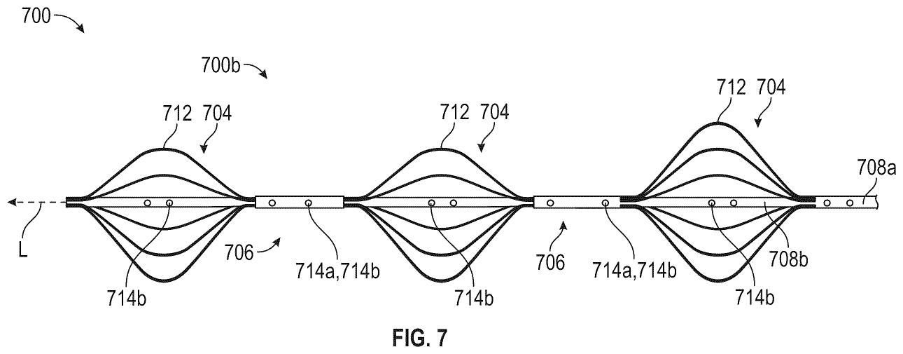

FIG. 7, for example, shows a side view of the distal portion 700b of a device 700 comprising a plurality of mechanical engagement regions 704 according to some embodiments of the present technology. The features of device 700 may be generally similar to those of device 600. However, device 700 may also be configured to supply negative pressure to the treatment site from within a plurality of expandable members 712 alternatively or in addition to from openings disposed along a plurality of aspiration regions 706. In some embodiments, the device 700 includes an outer tubular body 708a comprising a first plurality of sidewall openings 714a and an inner tubular body 708b comprising a second plurality of sidewall openings 714b slidably disposed therein. In this embodiment, at least some of the second plurality of openings 714b can be exposed to the treatment site when one or more of the expandable members 712 is in the expanded state. Additionally or alternatively, the first and second plurality of sidewall openings 714a, 714b may be configured to align as described previously herein, thereby resulting in aspiration between at least some of the plurality of mechanical engagement regions 704. In some embodiments, aspiration is supplied from locations along a full length of the distal portion 700b.

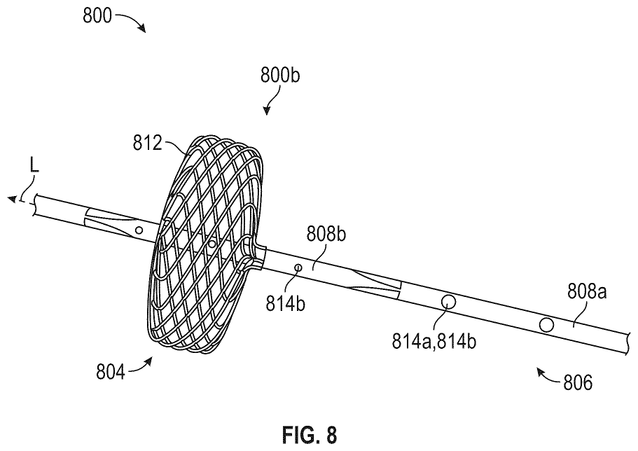

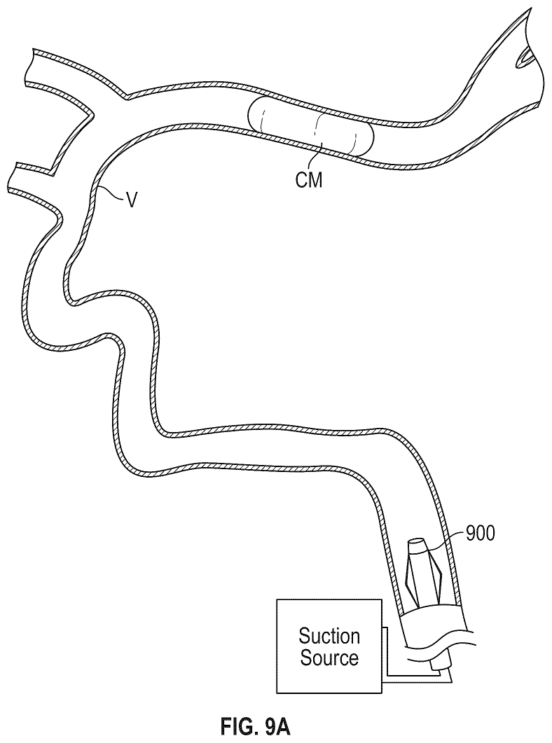

FIG. 8 shows a side view of the distal portion 800b of a device 800 according to some embodiments of the present technology. The device 800 can comprise features generally similar to any other embodiments described herein, differing in that the expandable member 812 of the mechanical engagement region 804 comprises a mesh element. In some embodiments, the mesh element is a high-density mesh. Additionally or alternatively, the expandable member 812 may comprise a stent, a braid, a plurality of struts, or another stent-like structure. In some embodiments, the mechanical engagement region 804 comprises a separate structure coupled to an outer tubular body 808a and/or an inner tubular body 808b, although the mechanical engagement region may also be integrally formed with at least a portion of device 800. Aspiration may be supplied from a first plurality of openings 814a disposed along a length of the outer tubular body 808a and/or from a second plurality of openings 814b disposed along a length of the inner tubular body 808b as described elsewhere herein. The first and second tubular bodies 808a, 808b may be configured to axially translate relative to one another. The expandable member 812 may be capable of axial movement in response to the relative axial translation of the first and second tubular bodies 808a, 808b. In some embodiments, the axial movement causes the expandable member 812 to transition from a collapsed state to an expanded state. In some embodiments, the expandable member 812 is configured to self-expand when in position at a treatment site.

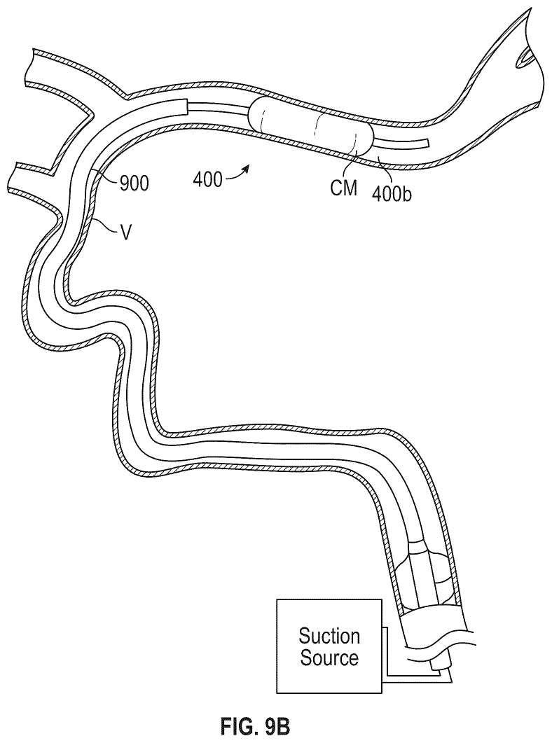

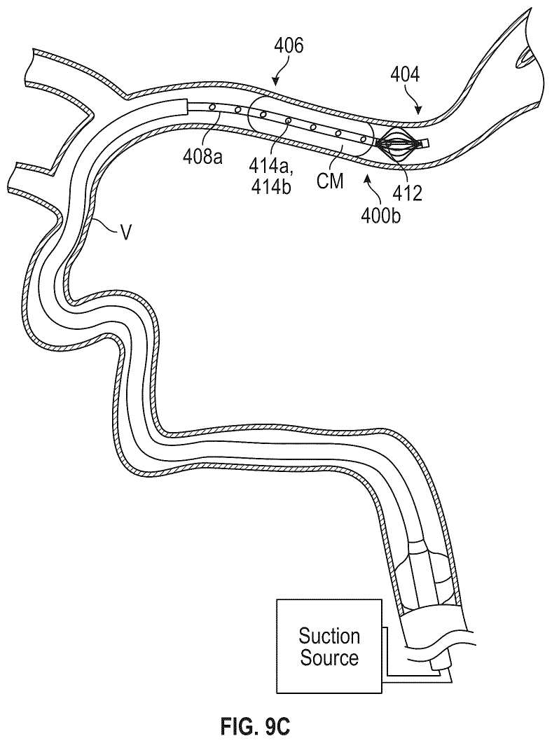

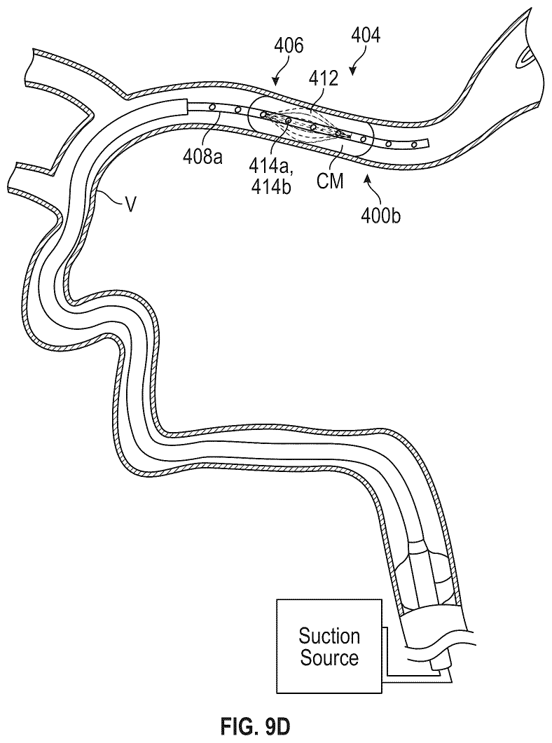

FIGS. 9A-9D illustrate exemplary methods of use of the devices of the present technology. In some embodiments, a device, for example the device 400 as shown in FIG. 4, may be introduced into the vasculature as shown in FIG. 9A. In some embodiments, the device 400 may be inserted through a larger tubular device 900; for example, a guide catheter or an aspiration catheter. As shown in FIG. 9B, the distal portion 400b of the device 400 may be advanced distally over a guidewire (not shown) through the clot material CM and/or between the clot material CM and the vessel wall. After the distal portion 400b is in position at the treatment site within vessel V, the expandable member 412 can be transitioned from the collapsed configuration to the expanded configuration. This transition may be achieved via retraction of the larger tubular device 900, self-expansion of the expandable member 412, relative axial motion between outer tubular body 408a and inner tubular body 408b, or any other suitable means. The expandable member 412 may be expanded proximally of, distally of, or within the clot material CM.

FIG. 9C shows an exemplary method in which the expandable member 412 is in its expanded configuration at a location within vessel V distal of the clot material CM. In such embodiments, the expandable member 412 acts as a distal protection element to prevent migration of detached clot material CM to distal vasculature.

FIG. 9D shows an exemplary method in which the expandable member 412 is in its expanded configuration within the clot material CM. Such embodiments may be advantageous for certain situations, for example, for clot material CM that is large or tightly lodged within the vessel V and difficult to retrieve via aspiration alone.

Following placement of the distal portion 400b, the guidewire may be removed (not shown). When the guidewire is removed, the plugs 418 remain in a sealed configuration as discussed above regarding FIGS. 3A and 3B. This inhibits fluid flow through the distal end of the device 400, thereby allowing greater negative pressure to be supplied through the sidewall openings 414a, 414b within the aspiration region 406.

As discussed previously, the first plurality of sidewall openings 414a of the outer tubular body 408a and the second plurality of sidewall openings 414b of the inner tubular body 408b may be substantially aligned when the expandable member 412 is in the expanded state. When the first and second plurality of sidewall openings 414a, 414b are at least partially aligned, fluid communication is permitted between the suction source and the treatment site via a lumen of the outer tubular body 408a and/or a lumen of the inner tubular body 408b. Accordingly, when negative pressure is supplied from the suction source when the expandable member 412 is in the expanded state, the aspiration region 406 may engage the clot material CM. Additionally or alternatively, the mechanical engagement region 404 may engage with the clot material CM. In some embodiments, the device 400 is moved proximally within vessel V while negative pressure is being supplied to enhance capture of the clot material CM by the mechanical engagement region 404. In some embodiments, at least one of the first and second plurality of sidewall openings 414a, 414b are configured to capture fragments of clot material CM that are too small to be captured by the mechanical engagement region 404.

While the exemplary methods above are discussed in regards to the device 400, a person having ordinary skill in the art will appreciate that these methods could readily be applied to other devices, including the other embodiments disclosed herein. The device 400 is included to illustrate a method of use of the present technology, but should not be construed as limiting these methods to any particular application. For example, similar methods could be employed to remove an obstruction from any other bodily lumen.

### CONCLUSION

Phrases such as an aspect, the aspect, another aspect, some aspects, one or more aspects, an implementation, the implementation, another implementation, some implementations, one or more implementations, an embodiment, the embodiment, another embodiment, some embodiments, one or more embodiments, a configuration, the configuration, another configuration, some configurations, one or more configurations, the subject technology, the disclosure, the present disclosure, other variations thereof and alike are for convenience and do not imply that a disclosure relating to such phrase(s) is essential to the subject technology or that such disclosure applies to all configurations of the subject technology. A disclosure relating to such phrase(s) can apply to all configurations, or one or more configurations. Such disclosure can provide one or more examples. A phrase such as an aspect can refer to one or more aspects and vice versa, and this applies similarly to other phrases.

Any implementation described herein as an “example” is not necessarily to be construed as preferred or advantageous over other implementations. Furthermore, to the extent that the term “include,” “have,” or the like is used in the description or the claims, such term is intended to be inclusive in a manner similar to the term “comprise” as “comprise” is interpreted when employed as a transitional word in a claim.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.”

Although many of the embodiments are described above with respect to systems, devices, and methods for treating vessel obstructions, the technology is applicable to other applications and/or other approaches. Moreover, other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above with reference to FIGS. 1-17.

The descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

As used herein, the terms “generally,” “substantially,” “about,” and similar terms are used as terms of approximation and not as terms of degree, and are intended to account for the inherent variations in measured or calculated values that would be recognized by those of ordinary skill in the art.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

The present technology is illustrated, for example, according to various aspects described below. Various embodiments of aspects of the present technology are described as numbered Examples (1, 2, 3, etc.) for convenience. These are provided as exemplary embodiments and do not limit the present technology. It is noted that any of the dependent Examples may be combined in any combination, and placed into a respective independent Example. The other Examples can be presented in a similar manner.

Example 1. A device for removing clot material from a vessel, comprising:
a tubular body defining a lumen configured to be fluidically coupled to a suction source, the tubular body comprising:
   an aspiration region comprising a plurality of sidewall openings in fluid communication with the lumen; and
   a mechanical engagement region disposed distal to the aspiration region, the mechanical engagement region comprising an expandable member configured to expand radially outwardly,
   wherein the expandable member is integrally formed with the tubular body.

Example 2. The device of any of the Examples herein, wherein the expandable member comprises a plurality of filaments separated by slits in the tubular body

Example 3. The device of any of the Examples herein, wherein, when the expandable member is in a collapsed state, an outer diameter of the tubular body is substantially constant along the aspiration region and the mechanical engagement region.

Example 4. The device of any of the Examples herein, wherein the tubular body is configured to be slidably advanced over a guidewire to a treatment site within a blood vessel, wherein the device further comprises a one-way valve disposed distal to the aspiration region, and wherein the one-way valve is configured to permit the guidewire to be slidably advanced therethrough and to inhibit flow of fluid therethrough after the guidewire is removed from the one-way valve.

Example 5. The device of any of the Examples herein, wherein the tubular body comprises an outer tubular body and the sidewall openings comprise first sidewall openings, and wherein the device further comprises an inner tubular body configured to be slidably disposed within the lumen of the outer tubular body, the inner tubular body comprising a plurality of second sidewall openings configured to be aligned with the first sidewall openings.

Example 6. The device of any of the Examples herein, wherein expansion of the expandable member causes the outer tubular body to move axially relative to the inner tubular body, and wherein the plurality of second sidewall openings are aligned with the first sidewall openings after the axial movement.

Example 7. The device of any of the Examples herein, wherein the mechanical engagement region is a first mechanical engagement region and the expandable member is a first expandable member, wherein the tubular body further comprises a second mechanical engagement region disposed proximal to the aspiration region, the second mechanical engagement region comprising a second expandable member configured to expand radially outwardly, and wherein the second expandable member is integrally formed with the tubular body.

Example 8. The device of any of the Examples herein, wherein the aspiration region is a first aspiration region and the plurality of sidewall openings is a first plurality of sidewall openings, the tubular body further comprising a second aspiration region disposed proximal to the second mechanical engagement region, the second aspiration region comprising a second plurality of sidewall openings in fluid communication with the lumen.

Example 9. A device for removing clot material from a vessel, comprising:
a first tubular body defining a first lumen, the first tubular body comprising: an aspiration region comprising a first plurality of sidewall openings; and
   a mechanical engagement region disposed distal to the aspiration region, the mechanical engagement region comprising an expandable member configured to expand radially outwardly; and
a second tubular body configured to be slidably disposed within the first lumen, the second tubular body defining a second lumen configured to be fluidically coupled to a suction source, the second tubular body comprising a second plurality of sidewall openings, wherein after expansion of the expandable member, at least some of the first plurality of sidewall openings are aligned with and in fluid communication with at least some of the second plurality of sidewall openings.

Example 10. The device of any of the Examples herein, wherein the first plurality of sidewall openings and second plurality of sidewall openings are not aligned when expandable member is in an unexpanded state.

Example 11. The device of any of the Examples herein, wherein the expandable member comprises a plurality of filaments separated by slits in the first tubular body.

Example 12. The device of any of the Examples herein, wherein, when the expandable member is in a collapsed state, an outer diameter of the first tubular body is substantially constant along the aspiration region and the mechanical engagement region.

Example 13. The device of any of the Examples herein, wherein the device further comprises a one-way valve disposed distal to the aspiration region, and wherein the one-way valve is configured to permit a guidewire to be slidably advanced therethrough and to inhibit flow of fluid therethrough after the guidewire is removed from the one-way valve.

Example 14. The device of any of the Examples herein, wherein the expandable member is integrally formed with first tubular body.

Example 15. The device of any of the Examples herein, wherein the mechanical engagement region is a first mechanical engagement region and the expandable member is a first expandable member, and wherein the first tubular body further comprises a second mechanical engagement region disposed proximal to the aspiration region, the second mechanical engagement region comprising a second expandable member configured to expand radially outwardly.

Example 16. The device of any of the Examples herein, further comprising a third plurality of sidewall openings, wherein the aspiration region is a first aspiration region and the first tubular body further comprises a second aspiration region disposed proximal to the second mechanical engagement region, and wherein third plurality of sidewall openings are disposed along the second aspiration region and in fluid communication with the second lumen.

Example 17. A method for removing clot material from a vessel, comprising: positioning a guidewire at a treatment site within the vessel;
advancing a device over the guidewire, wherein the device comprises a tubular body comprising a lumen in fluid communication with one or more sidewall openings and an expandable member disposed distal to at least one of the one or more sidewall openings;
removing the guidewire from the lumen;
expanding the expandable member; and
supplying negative pressure to the treatment site from a suction source in fluid communication with the lumen such that the clot material engages the one or more sidewall openings.

Example 18. The method of any of the Examples herein, wherein the device comprises a one-way valve disposed distal to the one or more sidewall openings and configured to permit the guidewire to be slidably advanced therethrough.

Example 19. The method of any of the Examples herein, wherein advancing the device over the guidewire comprises positioning the expandable member distal to at least a portion of the clot material.

Example 20. The method of any of the Examples herein, wherein the tubular body is an outer tubular body and the device further comprises an inner tubular body, and wherein expanding the expandable member slides the outer tubular body relative to the inner tubular body such that the one or more sidewall openings of the outer tubular body are aligned with one or more sidewall openings of the inner tubular body.

1. A device for removing clot material from a vessel, comprising:
a tubular body defining a lumen configured to be fluidically coupled to a suction source, the tubular body comprising:
an aspiration region comprising a plurality of sidewall openings in fluid communication with the lumen; and
a mechanical engagement region disposed distal to the aspiration region, the mechanical engagement region comprising an expandable member configured to expand radially outwardly,
wherein the expandable member is integrally formed with the tubular body.

2. The device of claim 1, wherein the expandable member comprises a plurality of filaments separated by slits in the tubular body.

3. The device of claim 1, wherein, when the expandable member is in a collapsed state, an outer diameter of the tubular body is substantially constant along the aspiration region and the mechanical engagement region.

4. The device of claim 1, wherein the tubular body is configured to be slidably advanced over a guidewire to a treatment site within a blood vessel, wherein the device further comprises a one-way valve disposed distal to the aspiration region, and wherein the one-way valve is configured to permit the guidewire to be slidably advanced therethrough and to inhibit flow of fluid therethrough after the guidewire is removed from the one-way valve.

5. The device of claim 4, wherein the tubular body comprises an outer tubular body and the sidewall openings comprise first sidewall openings, and wherein the device further comprises an inner tubular body configured to be slidably disposed within the lumen of the outer tubular body, the inner tubular body comprising a plurality of second sidewall openings configured to be aligned with the first sidewall openings.

6. The device of claim 5, wherein expansion of the expandable member causes the outer tubular body to move axially relative to the inner tubular body, and wherein the plurality of second sidewall openings are aligned with the first sidewall openings after the axial movement.

7. The device of claim 6, wherein the mechanical engagement region is a first mechanical engagement region and the expandable member is a first expandable member, wherein the tubular body further comprises a second mechanical engagement region disposed proximal to the aspiration region, the second mechanical engagement region comprising a second expandable member configured to expand radially outwardly, and wherein the second expandable member is integrally formed with the tubular body.

8. The device of claim 7, wherein the aspiration region is a first aspiration region and the plurality of sidewall openings is a first plurality of sidewall openings, the tubular body further comprising a second aspiration region disposed proximal to the second mechanical engagement region, the second aspiration region comprising a second plurality of sidewall openings in fluid communication with the lumen.

9. A device for removing clot material from a vessel, comprising:
a first tubular body defining a first lumen, the first tubular body comprising:
an aspiration region comprising a first plurality of sidewall openings; and
a mechanical engagement region disposed distal to the aspiration region, the mechanical engagement region comprising an expandable member configured to expand radially outwardly; and
a second tubular body configured to be slidably disposed within the first lumen, the second tubular body defining a second lumen configured to be fluidically coupled to a suction source, the second tubular body comprising a second plurality of sidewall openings, wherein after expansion of the expandable member, at least some of the first plurality of sidewall openings are aligned with and in fluid communication with at least some of the second plurality of sidewall openings.

10. The device of claim 9, wherein the first plurality of sidewall openings and second plurality of sidewall openings are not aligned when expandable member is in an unexpanded state.

11. The device of claim 9, wherein the expandable member comprises a plurality of filaments separated by slits in the first tubular body.

12. The device of claim 11, wherein, when the expandable member is in a collapsed state, an outer diameter of the first tubular body is substantially constant along the aspiration region and the mechanical engagement region.

13. The device of claim 12, wherein the device further comprises a one-way valve disposed distal to the aspiration region, and wherein the one-way valve is configured to permit a guidewire to be slidably advanced therethrough and to inhibit flow of fluid therethrough after the guidewire is removed from the one-way valve.

14. The device of claim 13, wherein the expandable member is integrally formed with first tubular body.

15. The device of claim 14, wherein the mechanical engagement region is a first mechanical engagement region and the expandable member is a first expandable member, and wherein the first tubular body further comprises a second mechanical engagement region disposed proximal to the aspiration region, the second mechanical engagement region comprising a second expandable member configured to expand radially outwardly.

16. The device of claim 15, further comprising a third plurality of sidewall openings, wherein the aspiration region is a first aspiration region and the first tubular body further comprises a second aspiration region disposed proximal to the second mechanical engagement region, and wherein third plurality of sidewall openings are disposed along the second aspiration region and in fluid communication with the second lumen.

17. A method for removing clot material from a vessel, comprising:
positioning a guidewire at a treatment site within the vessel;
advancing a device over the guidewire, wherein the device comprises a tubular body comprising a lumen in fluid communication with one or more sidewall openings and an expandable member disposed distal to at least one of the one or more sidewall openings;
removing the guidewire from the lumen;
expanding the expandable member; and
supplying negative pressure to the treatment site from a suction source in fluid communication with the lumen such that the clot material engages the one or more sidewall openings.

18. The method of claim 17, wherein the device comprises a one-way valve disposed distal to the one or more sidewall openings and configured to permit the guidewire to be slidably advanced therethrough.

19. The method of claim 18, wherein advancing the device over the guidewire comprises positioning the expandable member distal to at least a portion of the clot material.

20. The method of claim 19, wherein the tubular body is an outer tubular body and the device further comprises an inner tubular body, and wherein expanding the expandable member slides the outer tubular body relative to the inner tubular body such that the one or more sidewall openings of the outer tubular body are aligned with one or more sidewall openings of the inner tubular body.
